(19) United States
(12) Patent Application Publication
(10) Pub. No.: US 2021/0057102 A1
(43) Pub. Date: Feb. 25, 2021

(54) DENTAL HEALTH AND CARE SYSTEM

(71) Applicant: Yi-Chung CHEN, Taipei City (TW)

(72) Inventor: Yi-Chung CHEN, Taipei City (TW)

(21) Appl. No.: 16/784,972

(22) Filed: Feb. 7, 2020

(30) Foreign Application Priority Data
Aug. 23, 2019 (TW) 108130171

(51) Int. Cl. G16H 50/20; A61B 1/24; A61B 1/00; A61B 1/04; A61B 1/06; A46B 5/00; A46B 15/00; A46B 17/08; A46B 9/04; A46B 7/04; G16H 30/20; G16H 30/40; G16H 70/60; G16H 15/00; G06T 7/00; H04N 5/225

(52) U.S. Cl. CPC G16H 50/20; G06T 2207/30036; A61B 1/00016; A61B 1/04; A61B 1/06; A61B 1/00105; A46B 5/0095; A46B 15/0022; A46B 15/001; A46B 17/08; A46B 9/04; A46B 7/042; G16H 30/20; G16H 30/40; G16H 70/60; G16H 15/00; G06T 7/0014; H04N 5/2253; A46B 2200/1066; A61B 1/24

(57) ABSTRACT

A dental health and care system utilizes an image capturing module installed on a teeth cleaning device in order to capturing teeth images during the cleaning of teeth of a user. Teeth images can be submitted to a care service center to determine whether the oral care is proper performing, the teeth is cleaning, and any symptom is in the teeth thereof. In addition, it is able to further provide diagnosis recommendations containing medical treatment information in order to timely report to the care giver of the child, elderly or care receiver or to the user for improvement on the health habit or as outpatient reference. Professional consultation and recommendation can also be provided in order to achieve the effect of providing assistance to the user or the entire family members in their understanding of the dental health and care condition through daily teeth cleaning actions with ease.

### BACKGROUND OF THE INVENTION

### 1. Technical Field

The present invention relates to the application of dental cleaning and care, in particular, to a dental health and care system capable of assisting care givers, care receivers and users in taking care of dental cleaning and health condition.

### 2. Description of Related Art

Oral cavity and teeth are important organs for people to gain nutrients from food consumption. When the hygiene condition is poor, body health is likely to be affected and different level of dental diseases, such as decayed tooth and periodontal disease, due to gradually decayed tooth may also occur. Once a tooth is severely damaged due to disease, it is nearly impossible for such tooth to regenerate and recover. Accordingly, excellent health habit is extremely important.

To prevent occurrence of dental diseases, proper teeth cleaning method is imperative and the habit of excellent teeth cleaning habit is also important. Furthermore, dentists often recommend periodic dental examinations to patients such that any dental diseases can be discovered during the early stage of occurrence for early treatment, and improper teeth cleaning method can be corrected timely according to the improper cleaning location of teeth indicated in the examination result conducted by dentists, thereby achieving the objective of proper cleaning of teeth.

Nevertheless, due to the fast pace of modern urban living, people often overlook minor and trivial matters in their daily lives, and people tend to start to seek for medical assistance in dental examination or treatment when there is an issue in one's tooth; however, it may be too late to recover the damaged tooth. Presently, with regard to the dental or oral care, except for one's own personal care and awareness, there are no dental health and care system available in the market capable of providing assistance to care givers, care receivers and users in the care of dental cleaning and health. Particularly, when there are children, elderlies, physical or mental disabled in one's family, for care givers of care institutions, if there is a system capable of monitoring the oral health of a care receiving family, then the care givers are able to feel secured at work and the system is able to significantly overcome the oral health of general people by discovering problems early for improvement or treatment at early stage. With years of experience in medical practice, the inventor is committed to discovery problems and to overcome problems with solutions and improvements through dedicated researches in order to achieve the present invention.

### BRIEF SUMMARY OF THE INVENTION

The primary objective of the present invention is to provide a dental health and care system capable of using the daily teeth cleaning actions of a user to track and examine the cleaning condition and health condition, as well as to perform reporting immediately upon discovery of any problems, thereby allowing the user to timely examine the oral hygiene, to achieve prevention of dental diseases and to achieve the effect of early treatment upon initial stage of discovery of any problems.

Another objective of the present invention is to provide a dental health and care system capable of assisting care givers to automatically observe the teeth cleaning condition and health condition of care receivers (such as children and elderlies receiving care). In case where the teeth cleaning condition is found to be poor or teeth is examined to have any symptom, it is able to report to the care givers immediately in order to achieve the effect of protecting the dental care and protection against diseases for the entire family.

To achieve the foregoing objectives, the present invention provides a dental health and care system, comprising: a teeth cleaning device, an image capturing module, a data transmission module and a care service center. The teeth cleaning device is provided for a user to clean teeth. The image capturing module is installed on the teeth cleaning device and configured to capture teeth image of the user during teeth cleaning. The data transmission module is connected to the image capturing module and configured to transmit out teeth image data captured by the image capturing module. The care service center is connected to the data transmission module via an internet network in order to receive the teeth image data transmitted by the data transmission module. The care service center mainly comprises a user database, a intelligent health examination module, a diagnosis recommendation module and a reporting module. The user database is used for recording user data and the teeth image data. The intelligent health examination module is connected to the user database, and configured to examine the teeth image of the user and to analyze and compare the teeth image of the user based on a sample database stored with a plurality of learned symptom sample cases, thereby determining a teeth cleaning condition of the user and examining whether there is a symptom in teeth of the user. The diagnosis recommendation module is connected to the user database and the intelligent health examination module, and configured to provide at least one diagnosis recommendation from a medical resource database saved with a plurality of professional medical information corresponding to various symptoms according to the symptom and the user data. The reporting module is connected to the user database, the intelligent health examination module and the diagnosis recommendation module, and configured to report the teeth cleaning condition of the user or the symptom discovered during examination and the diagnosis recommendation to a predefined electronic device.

According to the aforementioned system architecture, wherein the teeth cleaning device comprises a control unit connected to the image capturing module, and the control unit is configured to further adjust and control an image capturing timing of the image capturing module.

According to the aforementioned system architecture, wherein the care service center further comprises a correction module; the correction module is connected to the intelligent health examination module and provided for a professional physician to correct the teeth cleaning condition determined or the symptom examined by the intelligent health examination module.

According to the aforementioned system architecture, wherein the image capturing module comprises a camera unit for image capturing and a lighting unit for providing illumination.

According to the aforementioned system architecture, wherein the teeth cleaning device is a toothbrush comprising a grip portion and a brush portion, and the brush portion is detachably attached onto the grip portion.

According to the aforementioned system architecture, wherein the image capturing module is installed on the brush portion and is located at an inner side of the brush portion having bristles or at an outer side of the brushing portion opposite from the bristles.

According to the aforementioned system architecture, wherein the image capturing module is installed at one end of the grip portion adjacent to the brush portion, and is located at one side identical to or opposite from the side of the brush portion having the bristles.

According to the aforementioned system architecture, wherein the teeth cleaning device further comprises a base for receiving the grip portion, and the data transmission module is installed on the base.

According to the aforementioned system architecture, wherein the electronic device is a mobile device used by the user, and the data transmission module is installed on the mobile device.

According to the aforementioned system architecture, wherein the teeth cleaning device further comprises a notification unit for notifying a replacement of the bristles. It is able to utilize an accumulative counting module built inside the control unit in order to accumulatively count the number of times of use and the period of use, such that when it reaches a predefined use standard, the notification unit is activated. Alternatively, the care service center is able to identify and determine the condition of bristles from the teeth image, such that when any improper condition (such as bristle deformation or deflection) occurs, it is able to activate the notification unit or report to the electronic device.

To further describe the aforementioned objectives, technical effects and technical features of the present invention in greater detail, please refer to the following accompanied drawings and description.

### BRIEF DESCRIPTION OF THE SEVERAL VIEWS OF THE DRAWINGS

FIG. 1 is an architecture schematic view of the dental health and care system of the present invention.

FIG. 2 is a schematic view showing a preferred embodiment of the present invention.

FIG. 3 is a schematic view showing another preferred embodiment of the present invention.

FIG. 4 is a schematic view showing still another preferred embodiment of the present invention.

FIG. 5 is a schematic view showing still another preferred embodiment of the present invention.

FIG. 6 is a schematic view showing still another preferred embodiment of the present invention.

FIG. 7 is a schematic view showing still another preferred embodiment of the present invention.

### DETAILED DESCRIPTION OF THE INVENTION

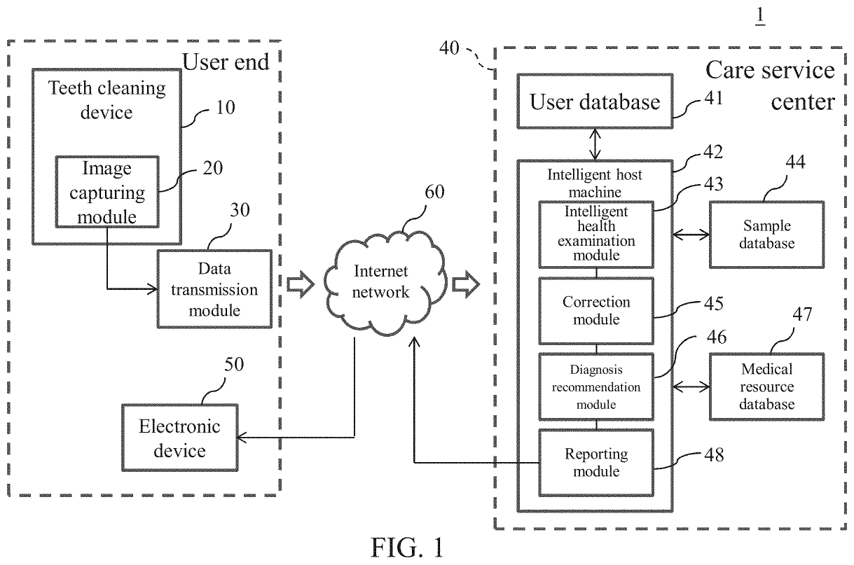

Please refer to FIG. 1. As shown in the figure, a dental health and care system 1 of the present invention mainly comprises two parts of a user end and a care service center 40. The user end mainly comprises a teeth cleaning device 10, a data transmission module 30 and an electronic device 50. The care service center 40 comprises a user database 41, an intelligent host machine 42, a sample database 44 and a medical resource database 47.

The teeth cleaning device 10 is mainly provided for a user to clean teeth, and it can be a tool of toothbrush or electric toothbrush for cleaning the teeth. In the present invention, an image capturing module 20 is installed on the teeth cleaning device 10, and the image capturing module 20 is used to capture teeth image during the cleaning of teeth of the user. The data transmission module 30 is mainly connected to the image capturing module 20 and configured to perform transmission of the teeth image data of the user captured by the image capturing module 20 through the internet network 60. In practice, the information transmission module 30 can use wired or wireless method to connect to the image capturing module 20, and the method of WIFI or Bluetooth etc. can be used for the image data transmission.

The care service center 40 is mainly connected to the data transmission module 30 via the internet network 60 in order to receive the teeth image data of the user outputted by the data transmission module 30. The user database 41 is used to record the user data and the teeth image data. The intelligent host machine 42 is able to perform determination based on the teeth image data in order to provide the service of dental health and care to the user. It mainly comprises an intelligent health examination module 43, a correction module 45, a diagnosis recommendation module 46 and a reporting module 48. The intelligent health examination module 43 is at least connected to the user database 41, and it is mainly for examining the teeth image of the user and analyzing and comparing the teeth image of the user based on the sample database 44 (it mainly cooperates with the intelligent health examination module 43 and provides a certain quantity of sample cases stored with a plurality of symptoms, and is also equipped with the functions of learning and expansion of cases) in order to determine the cleaning condition (such as comparison with the previous teeth image record of the user) of the teeth of the user as well as to examine whether there is any symptom (such as comparison with the symptom sample cases) in the teeth of the user. The correction module 45 is connected to the intelligent health examination module 43 and is provided for a professional physician to examine the determination or examination result of the intelligent health examination module 43 in order to correct any errors in the condition or symptom determined. Furthermore, cases with confirmed symptoms can also be recorded in the sample database 44. The diagnosis recommendation module 46 is at least connected to the user database 41 and the intelligent health examination module 43. When the intelligent health examination module 43 examines to find a symptom, it is able to provide at least one diagnosis recommendation (such as providing similar and appropriate medical information according to the symptom examined and the residence or workplace of the user) from the medical resource database 47 (mainly stores corresponding medical information of professional physicians of different departments, hospitals or clinics based on various dental symptoms) according to the symptom and the user data base. The reporting module 48 is at least connected to the user database 41, the intelligent health examination module 43 and the diagnosis recommendation module 46, such that when the teeth cleaning condition of the user is determined to have issues (such as insufficient number of cleaning, improper cleaning etc.), it is able to immediately report to the electronic device 50 predefined by the user, or when the teeth of the user is examined to find any symptom (such as decayed tooth, periodontal disease, malalignment of teeth etc.), it is able to generate a reporting message incorporating the symptom and the diagnosis recommendation therein for reporting to the electronic device 50 predefined by the user.

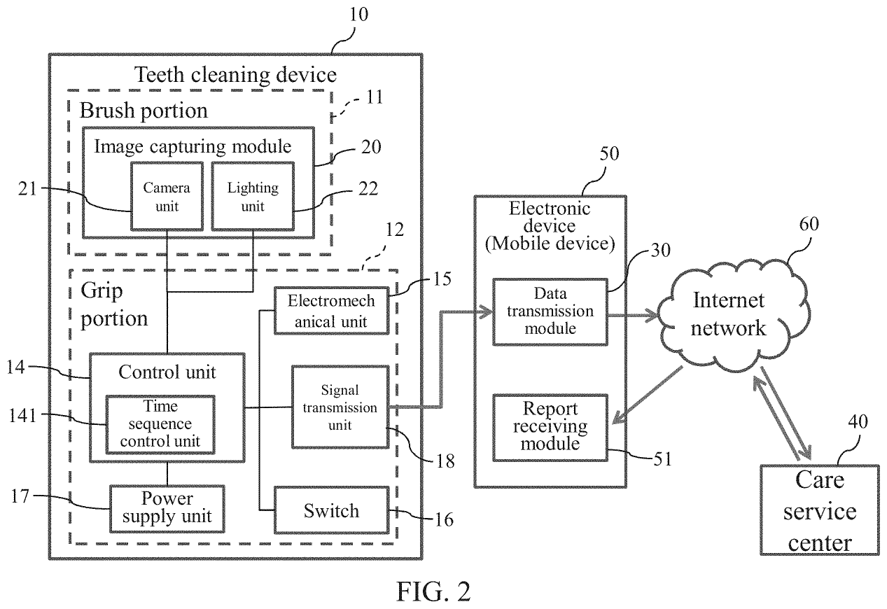

In a feasible embodiment, as shown in FIG. 2, the teeth cleaning device 10 refers to an electric toothbrush as an example for illustration, and typically comprises a brush portion 11 and a grip portion 12. The brush portion 11 is detachably attached onto the grip portion 12. The grip portion 12 includes a control unit 14, an electromechanical element 15, a switch 16, a power supply unit 17, and a signal transmitting unit 18 connected to each other. The brush portion 11 includes the image capturing module 20 installed thereon and it can be electrically connected to the control unit 14 when the brush portion 11 is attached onto the grip portion 12. The brush portion 11 includes bristles, and one side of the brush portion with the bristles is defined as the inner side, and the other side opposite from the bristles is defined as the outer side. In this embodiment, the image capturing module 20 can be installed on either the inner side or the outer side, and it can be located at an appropriate distance away from the perimeter of the bristles (such as the front end or rear end) or at a position on the outer side corresponding to the bristles, such that when user is cleaning the teeth, it is able to directly capture the teeth image inside the oral cavity of the user. Through the control unit 14 and the signal transmitting unit 18, the teeth image can be transmitted to the data transmission module 30.

In this embodiment, the electronic device 50 utilizes a mobile device commonly used by the user, and it also uses a program and a built-in transmission component as the data transmission module 30 and the report receiving module 51. The information transmission module 30 is used to transmit the teeth image data sent from the teeth cleaning device 10 to the care service center 40 via the internet network 60. Once the care service center 40 determines the cleaning condition or symptom of the teeth, it then feeds back the reporting message indicating the issue found to the report receiving module 51 of the electronic device 50 in order to allow the user to timely understand the teeth care condition.

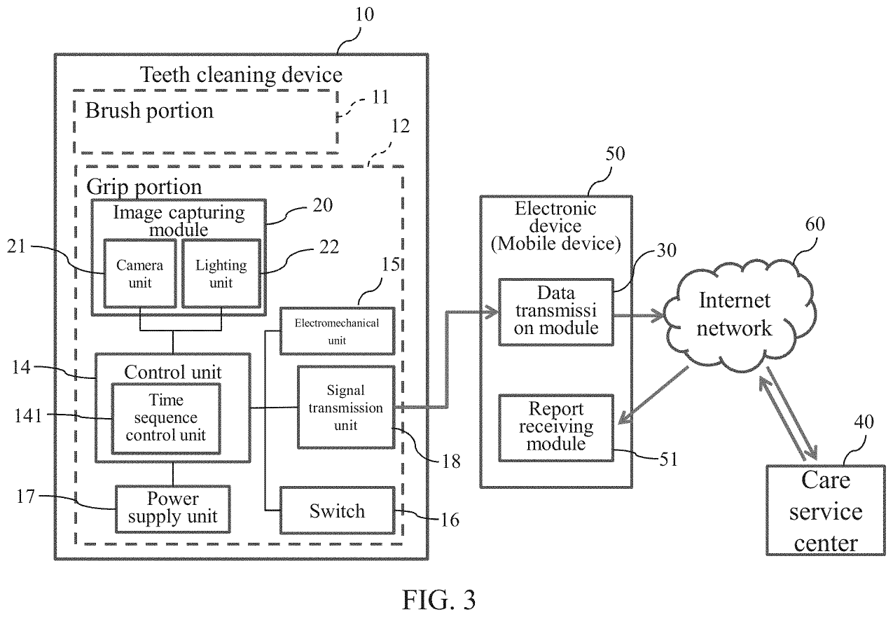

In another feasible embodiment, as shown in FIG. 3, in which an electric toothbrush is also used as an example for illustration. The teeth cleaning device 10 comprises a brush portion 11 and a grip portion 12, and the brush portion 11 is detachably attached onto the grip portion 12. In this embodiment, the grip portion 12 includes a control unit 14, an electromechanical element 15, a switch 16, a power supply unit 17, a signal transmitting unit 18 and the image capturing module 20 installed thereon and connected to each other. The image capturing module 20 can be installed at one end of the grip portion adjacent to the brush portion, and it can be located on one side identical to or opposite from the bristles of the brush portion (at the inner side identical to the side of the bristles or at the outer side opposite from the side of the bristles) in order to perform camera shooting at an appropriate angle. Therefore, during the cleaning of teeth of the user, it is able to photograph the oral cavity of the user in order to capture teeth images inside the oral cavity of the user, following which the control unit 14 and the signal transmitting unit 18 are able to transmit the teeth image data to the data transmission module 30. The electronic device 50 utilizes a mobile device commonly used by the user, and the data transmission module 30 can be installed therein in order to transmit the teeth image data sent from the teeth cleaning device 10 to the care service center 40 via the internet network 60. Once the care service center 40 determines the cleaning condition or symptom of the teeth, it then feeds back the reporting message indicating the issue found to the report receiving module 51 of the electronic device 50 in order to allow the user to timely understand the teeth care condition.

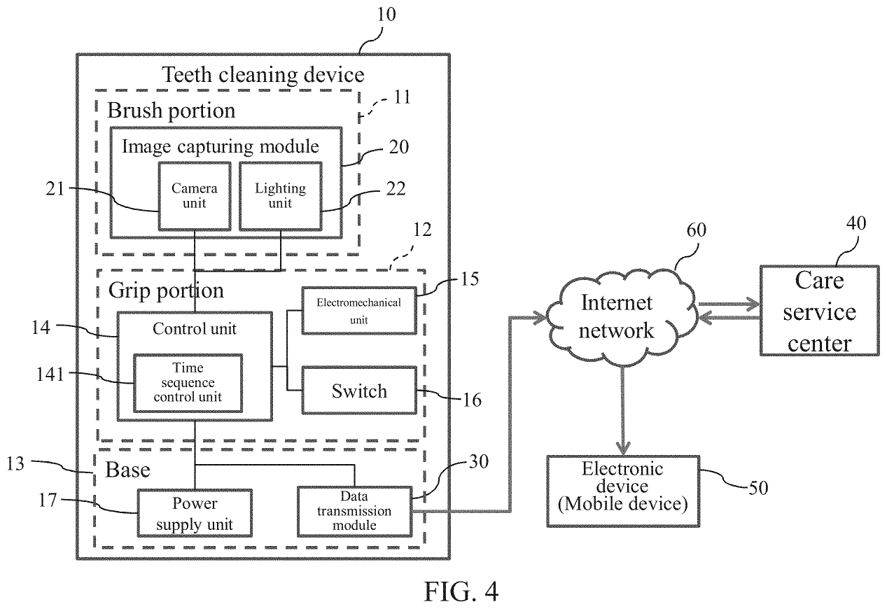

In still another feasible embodiment, as shown in FIG. 4, the teeth cleaning device 10 also utilizes an electric toothbrush as an example for illustration, but it is now divided into three parts of a brush portion 11, a grip portion 12 and a base 13. The brush portion 11 is detachably attached onto the grip portion 12, and the base 13 is provided to receive the grip portion 12 thereon. The grip portion 12 includes a control unit 14, an electromechanical element 15 and a switch 16 installed thereon and connected to each other. The image capturing module 20 is installed on the brush portion 11, such that when the brush portion 11 is attached onto the grip portion 12, it is electrically connected to the control unit 14. The brush portion 11 includes bristles installed thereon, and one side thereof having the bristles is defined as the inner side, and the other side opposite from the bristles is defined as the outer side. The image capturing module 20 can be installed on either the inner side or the outer side, and it can be located at an appropriate distance away from the perimeter of the bristles (such as front end or rear end) or at a position on the outer side corresponding to the bristles, such that when user is cleaning the teeth, it is able to directly capture the teeth image inside the oral cavity of the user. The base 13 includes a power supply unit 17 installed thereon. The base 13 can be a charging base (with a charge storing unit further installed on the grip portion 12) connected wirelessly with the grip portion 12, or it can be a charging base connected to the grip portion 12 in a wired method. Furthermore, in this embodiment, the data transmission module 30 is installed on the base 13 and is used to transmit the teeth image data captured by the image capturing module 20 during the teeth cleaning of the user to the care service 40 via the internet network 60. Once the care service center 40 determines the cleaning condition or symptom of the teeth, it then feeds back the reporting message indicating the issue found to the report receiving module 51 of the electronic device 50 in order to allow the user to timely understand the teeth care condition.

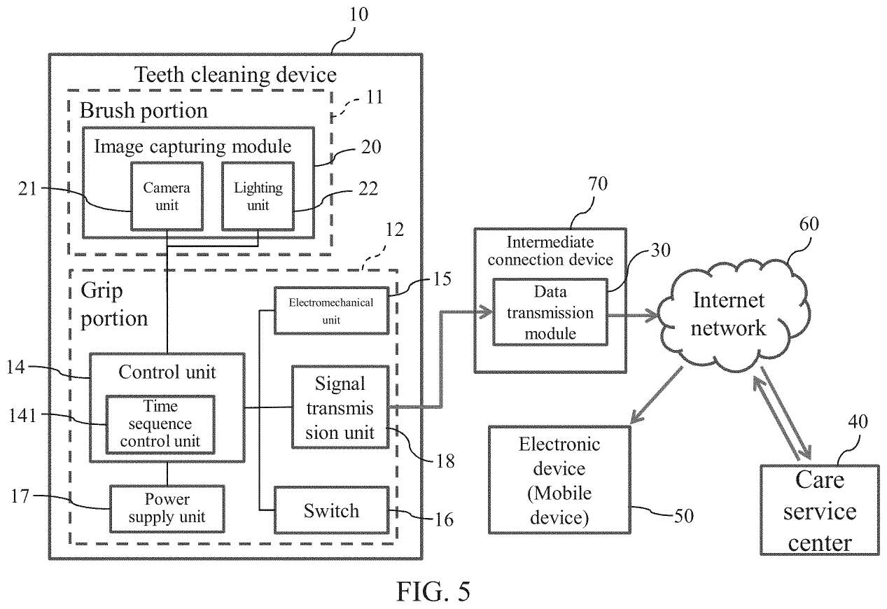

In still another feasible embodiment, as shown in FIG. 5, the assembly configuration of the teeth cleaning device 10 is similar to that of the embodiment shown in FIG. 2; therefore, similar configurations are omitted hereafter. Nevertheless, the data transmission module 30 is installed on an intermediate connection device 70. The intermediate connection device 70 can be a household device, such as a gateway or router etc., and it is able to transmit the teeth image data sent from the teeth cleaning device 10 to the care service center 40 via the internet network 60. Once the care service center 40 determines the cleaning condition or symptom of the teeth, it then feeds back the reporting message indicating the issue found to the electronic device 50 (such as a mobile device commonly used by the user) in order to allow the user to timely understand the teeth care condition.

In a feasible embodiment, the brush portion 11 of the teeth cleaning device 10 can be a commonly seen detachable structure, and it typically comprises a connecting rod, and through such connecting rod, it is able to detachably attached onto the grip portion 12. Nevertheless, the brush portion 11 can be configured to have a brush mounting structure in order to use the mounting or locking methods to be detachably attached onto a supporting rod extended from the grip portion 12. Accordingly, it is able to facilitate the arrangement of the image capturing module 20 on the teeth cleaning device 10, thereby satisfying the needs of various designs and uses.

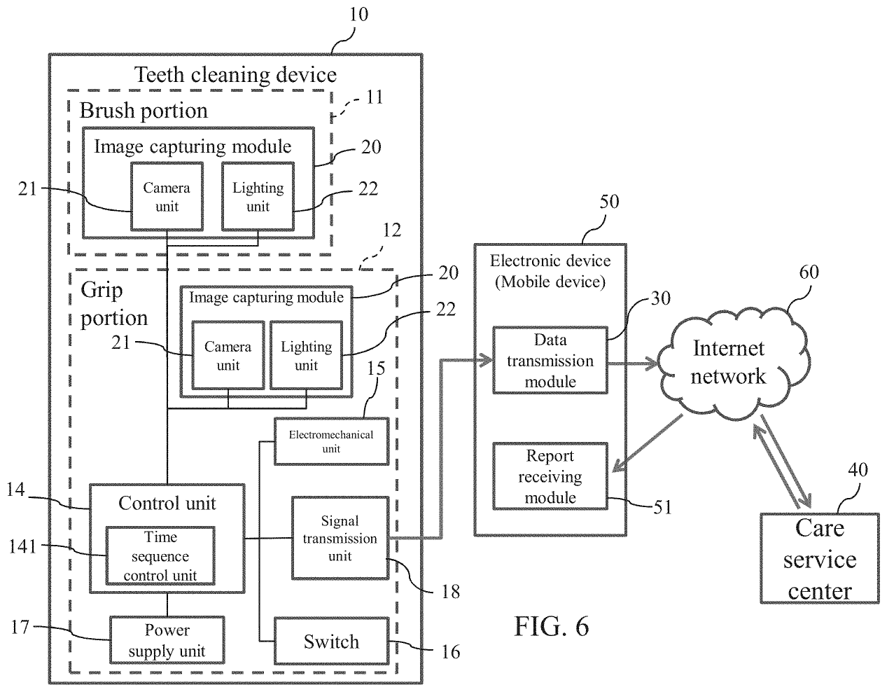

In addition, the quantity of the image capturing module 20 installed on the teeth cleaning device 10 can be one or a plurality of the modules, and the installation locations can also depend upon the actual needs. For example, the inner side and the outer side of the brush portion 11 can be installed with an image capturing module 20 respectively. Alternatively, the grip portion 12 of the brush portion 11 can be installed with one or a plurality of image capturing modules 20 (as the embodiment shown in FIG. 6, at least one of the image capturing module 20 is installed at the brush portion 11 and the grip portion 12 respectively, and other components are the same as the ones of the embodiment shown in FIG. 2; details thereof are omitted hereafter) in order to obtain more complete teeth images for transmitting to the care service center 40 to make determination of greater accuracy. In practice, the image capturing module 20 can comprise a camera unit 21 for capturing images and a lighting unit 22 for providing illumination. Accordingly, during the capturing of the teeth images, greater lighting can be provided in order to ensure the quality of the teeth images. In addition, the image capturing module 20 can select the camera filming mode or photo taking mode. The control unit 14 can further include a time sequence control module 141 for adjusting the image capturing timing of the image capturing module 20, especially that under the photo taking mode, for example, a photo image can be taken every 3 seconds or 5 seconds during the teeth cleaning of the user.

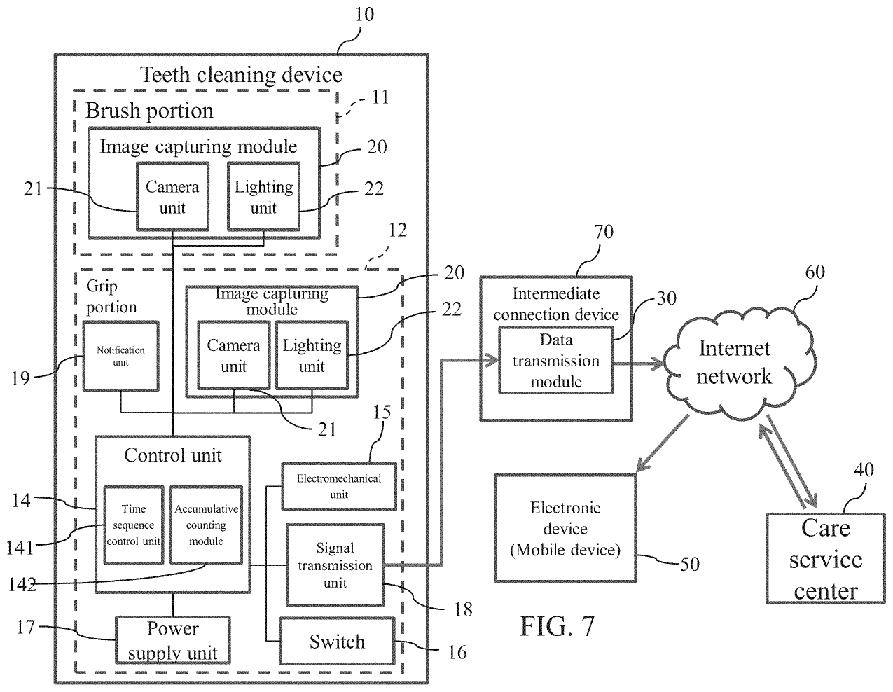

Furthermore, the dental health and care system 1 of the present invention can be further provided with the function of automatic notification for replacement of bristles. Such function can be achieved by installing corresponding component on the teeth cleaning device 10 for automatic notification, or relevant data can be provided to the care service center 40 for determination, followed by providing notification to the user. A method of the automatic notification of the teeth cleaning device 10 is as shown in FIG. 7, and the teeth cleaning device 10 includes the components of two detachable parts of the brush portion 11 and grip portion 12, and at least one image capturing module 20, a control unit 14, an electromechanical element 15, a switch 16, a power supply unit 17, a signal transmitting unit 18 etc. The control unit 14 includes a time sequence control module 141 and further includes an accumulative counting module 142 to accumulatively count the number of times of use and the period of use, such that when the accumulated value reaches a predefined use standard, the notification unit is activated. Alternatively, the care service center is able to control and active a notification unit 19 in order to notify the user that the bristles have been used for a long time and it is necessary to replace the bristles. After the bristles are replaced, a notification unit 19 can be turned off via manual or automatic method. The notification unit 19 can be an indicator lamp exposed at the grip portion 12 or it can be a buzzer or speaker capable of generating alarm sound or voice. In addition, the care service center 40 can also determine the notification method (not shown in the drawings; please refer to FIG. 1). When the intelligent host machine 42 determines the cleaning condition or symptom of the teeth, it is able to identify the information of the bristles from the teeth image altogether in order to provide such information to a bristle inspection module further installed on the intelligent host machine 42 in order to inspect the usage change of the bristles. When the bristles are found to have deformation or deflection etc. it is able to feed back a message via the reporting module 48, and the notification unit 19 of the teeth cleaning device 10 can be controlled to notify the user to replace the bristles. Alternatively, a report message indicating the necessity for bristle replacement can be generated, and the reporting module 48 then reports such message to the electronic device 50 predefined by the user in order to notify the user to replace the bristles. In practice, if necessary, this system can also report the report message of the bristles to the device supplier or relevant medical care personnel to make appropriate responsive actions.

In a feasible embodiment, the user operating the teeth cleaning device 10 can be identical to or different from the user receiving the report message. If the users are the same, then the use can use the dental health and care system 1 of the present invention to look after the health condition of his or her own teeth. In addition, when the cleaning condition is poor or when there is any symptom in the teeth, the user is able to receive report message in order to improve the personal health habit, or the user can further review the diagnosis recommendation in the report message in order to seek medical assistance and treatment immediately, thereby achieving the objective of health and care. If the aforementioned users are different from each other, such as the relationship between children or elderly and the care giver, then the dental health and care system 1 of the present invention is able to assist the care giver to take care of the dental health condition of the entire family through receiving report messages timely and to urge family members for improvement and medical treatment at early stage.

In view of the above, the present invention is able to achieve the technical effects of providing dental health and care to an individual or entire family members, and warnings can be provided timely without affecting the daily lives, thereby achieving the effects of improving dental hygiene and disease prevention. The present invention is of novelty and inventive step, and a patent application is hereby submitted according to the laws. It shall be understood that the aforementioned description is provided for the purpose of illustrating the preferred embodiments of the present invention only, and any changes, modifications, alternations or equivalent replacements of the technical means and scope of the present invention shall be within the scope of the claims of the present invention.

What is claimed is:

1. A dental health and care system, comprising:
a teeth cleaning device provided for a user to clean teeth:
an image capturing module installed on the teeth cleaning device and configured to capture teeth image of the user during teeth cleaning;
a data transmission module connected to the image capturing module and configured to transmit out teeth image data captured by the image capturing module;
a care service center connected to the data transmission module via an internet network in order to receive the teeth image data transmitted by the data transmission module, and comprising:
a user database for recording user data and the teeth image data;
an intelligent health examination module connected to the user database, and configured to examine the teeth image of the user and to analyze and compare the teeth image of the user based on a sample database stored with a plurality of learned symptom sample cases, thereby determining a teeth cleaning condition of the user and examining whether there is a symptom in teeth of the user;
a diagnosis recommendation module connected to the user database and the intelligent health examination module, and configured to provide at least one diagnosis recommendation from a medical resource database saved with a plurality of professional medical information corresponding to various symptoms according to the symptom and the user data; and
a reporting module connected to the user database, the intelligent health examination module and the diagnosis recommendation module, and configured to report the teeth cleaning condition of the user or the symptom discovered during examination and the diagnosis recommendation to a predefined electronic device.

2. The dental health and care system according to claim 1, wherein the teeth cleaning device comprises a control unit connected to the image capturing module, and the control unit is configured to further adjust and control an image capturing timing of the image capturing module.

3. The dental health and care system according to claim 1, wherein the care service center further comprises a correction module; the correction module is connected to the intelligent health examination module and provided for a professional physician to correct the teeth cleaning condition determined or the symptom examined by the intelligent health examination module.

4. The dental health and care system according to claim 2, wherein the image capturing module comprises a camera unit for image capturing and a lighting unit for providing illumination.

5. The dental health and care system according to claim 1, wherein the teeth cleaning device is a toothbrush comprising a grip portion and a brush portion, and the brush portion is detachably attached onto the grip portion.

6. The dental health and care system according to claim 5, wherein the image capturing module is installed on the brush portion and is located at an inner side of the brush portion having bristles or at an outer side of the brushing portion opposite from the bristles.

7. The dental health and care system according to claim 5, wherein the image capturing module is installed a location of the grip portion adjacent to the brush portion, and is located at one side identical to or opposite from the side of the brush portion having the bristles.

8. The dental health and care system according to claim 5, wherein the teeth cleaning device further comprises a base for receiving the grip portion, and the data transmission module is installed on the base.

9. The dental health and care system according to claim 1, wherein the electronic device is a mobile device used by the user, and the data transmission module is installed on the mobile device.

10. The dental health and care system according to claim 5, wherein the teeth cleaning device further comprises a notification unit for notifying a replacement of the bristles.

11. The dental health and care system according to claim 2, wherein the teeth cleaning device is a toothbrush comprising a grip portion and a brush portion, and the brush portion is detachably attached onto the grip portion.

12. The dental health and care system according to claim 11, wherein the image capturing module is installed on the brush portion and is located at an inner side of the brush portion having bristles or at an outer side of the brushing portion opposite from the bristles.

13. The dental health and care system according to claim 11, wherein the image capturing module is installed a location of the grip portion adjacent to the brush portion, and is located at one side identical to or opposite from the side of the brush portion having the bristles.

14. The dental health and care system according to claim 11, wherein the teeth cleaning device further comprises a base for receiving the grip portion, and the data transmission module is installed on the base.

15. The dental health and care system according to claim 4, wherein the teeth cleaning device is a toothbrush comprising a grip portion and a brush portion, and the brush portion is detachably attached onto the grip portion.

16. The dental health and care system according to claim 15, wherein the image capturing module is installed on the brush portion and is located at an inner side of the brush portion having bristles or at an outer side of the brushing portion opposite from the bristles.

17. The dental health and care system according to claim 15, wherein the image capturing module is installed a location of the grip portion adjacent to the brush portion, and is located at one side identical to or opposite from the side of the brush portion having the bristles.

18. The dental health and care system according to claim 15, wherein the teeth cleaning device further comprises a base for receiving the grip portion, and the data transmission module is installed on the base.

19. The dental health and care system according to claim 2, wherein the electronic device is a mobile device used by the user, and the data transmission module is installed on the mobile device.

20. The dental health and care system according to claim 4, wherein the electronic device is a mobile device used by the user, and the data transmission module is installed on the mobile device.
